(19) United States
(12) Patent Application Publication
(10) Pub. No.: US 2026/0283517 A1
(43) Pub. Date: Sep. 24, 2026

(54) SAMPLING DEVICE

(71) Applicant: IKASHMORE PTY LTD, New South Wales (AU)

(72) Inventor: Avtar Singh KASHMIRIAN, New South Wales (AU)

(21) Appl. No.: 19/475,920

(22) PCT Filed: Apr. 16, 2024
(86) PCT No.: PCT/AU2024/050361

§ 371 Date: Oct. 16, 2025

(30) Foreign Application Priority Data
Apr. 17, 2023 (AU) 2023901132

(51) Int. Cl. A61B 5/154; A61B 5/15

(52) U.S. Cl. CPC A61B 5/154; A61B 5/150221; A61B 5/150389; A61B 5/150732

(57) ABSTRACT

A sampling device adapted to connect in use a needle or blood bag tube with a sampling container; said device including a base member having a bottom end, a base rim, resilient extensions extending from the base rim, and an elongate boss having an aperture defining a longitudinal axis a spring adapted in use to be located at least partly within said chamber, about said boss and extending away from said bottom end a lower member having a flange and an aperture at least one leg extending away from said flange opposite to said collar and an angled protrusion extending outwards from the longitudinal axis and wherein the angled projection sits above the resilient extensions in a position wherein the needle or blood bag tube is not engaged with the sampling container an upper member having a flange in use operatively associated with the flange of said lower member.

### FIELD

The present invention relates to a sampling device and in particular to a device to facilitate the safe sampling of liquids from a needle or other such elongated tubular injection device.

### BACKGROUND

It is known to take samples of liquids from various locations, such as blood in a human for example, to allow researchers, doctors, medical technicians or the like to conduct testing of those samples. In the past, such testing has involved the use of an injection device such as a needle, withdrawing a sample of the liquid from a location or reservoir (such as the arm of a person) and expelling some of that liquid into a sampling container such as a bottle, vial, test tube, or the like. Due to the nature of such operations, the doctor, researcher or medical technician can inflict a needle stick injury on themselves or others. This can be compounded by the transference of blood, viruses and other materials.

To alleviate this, larger, safer injectors were created. However, such devices are excessively complicated, difficult to manufacture and very expensive to produce which greatly inhibits their use as it is common for locations where a large amount of sampling occurs such as blood banks, pathologists hospitals, universities or the like, to utilise a large number of needles. By utilising cheaper, simpler needles, with a sampling device or adapter fitted between the needle and the sampling container, needle stick injuries and transference could be reduced. It has however been found that such sampling devices have not been sufficiently engineered, are not tamper proof and still cause needle stick injuries and transference. Further, such devices are often complicated, can break easily and are too expensive. Accordingly, there is a need for a better sampling device/adapter which prevents needle stick injuries and transference occurring, whilst also being of simple construction, easy to manufacture and inexpensive.

To address this, existing devices have been developed that are able to receive a container with a set diameter or perimeter. When a container with a diameter or perimeter smaller or larger than the set diameter or perimeter is engaged with the device, the device stops the engagement of a piercing device with the device to allow collection of a sample in a sampling container.

Further, in the art of blood collecting a typical “tube-barrel holder” or also sometimes called “multi-sampling port” is used. It may also have a lid at the open end. The base typically has a boss with a female thread. Also used is the multi sample luer adaptor which consists of a plastic connector into which a steel needle is bonded and the needle is then covered with an elastomeric sheath. The sheath allows multiple sampling of the blood via the needle using different test or evacuated tubes. The sheath retracts over the open needle mouth when the test tube is retracted thus stopping flow of blood until another tube is inserted. Basically the sheath works like a valve. The needle in the sheath is normally a fixed size and does not vary with patients. The sheathed needle is always connected inside the tube barrel holder. The blunt end of the multi-sample luer adaptor is often tapered (hence called a luer) to connect to connectors with corresponding tapers. If a connection to a tube is required instead, then there may not be any taper on this end (and it will not be called a luer connector).

Often a “winged-needle” also called “butterfly” needle is used and connected via a tubing to a female luer connector. This luer connector can be connected to the male luer of the “multi-sample luer adaptor” mentioned above. The winged needle comes in various sizes. The multi sample luer adaptor for example can be screwed into the base of a tube-barrel holder. The winged needle is then used in a patients arm to extract blood. Sometimes instead of using “winged needles”, medical staff use a double needle assembly which has a sheathed needle connected to a longer hollow needle (that has no sheath). This can be screwed into a tube barrel holder and used to collect blood from patients as well. The sheath needle is screwed inside the tube barrel holder and the venipuncture needle is outside. The exposed needle comes in various sizes (gauge 20G, 21G, 22G etc). The sheathed needle is a fixed size and does not change. It is generally a heavier gauge so that it can penetrate elastomeric bungs or plugs.

Sometimes needles with a female luer are directly connected to the male luer of the “multi sample luer adaptor” which allows the tube barrel holder to be connected to a needle for venipuncture of patient. When venipuncture is performed on an arm with a needle the blood collects into an evacuated tube. The evacuated tube is fully inserted into the tube barrel holder so that the sheathed needle inside the tube barrel holder has penetrated the bung in the evacuated tube and thus allows the flow of blood from the vein into the tube. Sometimes the tube barrel holder can be connected to a “cannula” which is inserted into a vein. The tube barrel holder can be connected directly to tubing in a blood bag kit. In these situations the tube barrel holder is used to get blood samples from the bag and not directly from a patient's arm. Inside the tube barrel holder is the sheathed needle or multisample adaptor. The evacuated tubes or test tubes have a slight vacuum which allows blood to be “sucked” into the chamber and they come in various sizes generally 3 ml to 10 ml. In all cases the tube barrel holder must contain a multi sample needle (sheathed needle) which may be; connected to a second needle via a luer connection. Second needle can be normal or butterfly type; connected directly to a second needle with no luer connection; or connected directly to a tube from a blood bag with no luer connection.

There is a need for a device for taking samples of liquids with a piercing device such as a needle that provides a safety restraint to minimise the risk of a skin puncture on a user when using a variety of diameter/perimeter sample containers.

It is an object of this invention to substantially overcome or at least ameliorate one or more disadvantages of the prior art, or at least provide a useful alternative.

### SUMMARY OF INVENTION

Disclosed herein is a sampling device adapted to connect in use a needle or blood bag tube with a sampling container, the sampling device configured for movement between a neutral position and an active position, the sampling device including:
a base member including:
at least one base member sidewall to define a base chamber; and
a boss located within the chamber, the boss including a base member aperture configured for holding the needle in use;
a lower member configured for nesting at least partially within the base chamber, the lower member including:
a lower member aperture axially aligned with the base member aperture to receive the needle therethrough;
at least one lower member leg that extend from around the lower member aperture to engage the sampling container in use;
an upper member configured for axial movement toward and away from the base member, the upper member including:
at least one upper member sidewall to define an upper member chamber;
an upper member protrusion located within the upper member chamber, the upper member protrusion including an upper member aperture axially aligned with the base member aperture for the needle to pass therethrough; and
a spring seated on the boss, the spring configured for biasing the lower member away from the base member toward the neutral position,
wherein in the neutral position, the upper member protrusion is configured to shield the needle, and in the active position the needle is exposed from the upper member protrusion and shielded by the upper member chamber.

In one embodiment, the at least one lower member leg is configured to engage the sampling container when the sampling container is inserted into the upper member chamber.

In one embodiment, when the at least one lower member leg is seated on the sampling container, and the sampling device is moved toward the sampling container such that the sampling device moves from neutral position to the active position, the needle protrudes from the upper member protrusion.

In one embodiment, the sampling device is movable from the neutral position to the active position when the at least one lower member leg is seated on a shoulder of the sampling container.

In one embodiment, the lower member includes a flange, the at least one lower member leg extends from the flange.

In one embodiment, a longitudinal axis extends through the base member aperture, the lower member aperture and the upper member aperture.

In one embodiment, the upper member includes at least one slot adapted in use to receive the at least one lower member leg.

In one embodiment, at least one slot is transverse to the longitudinal axis to allow the at least one lower member leg to pass therethrough.

In one embodiment, the lower member includes at least one angled protrusion extending outwards from the longitudinal axis.

In one embodiment, the upper member includes at least one further slot arranged to receive the angled protrusion in use.

In one embodiment, the base member sidewall includes a ledge extending toward the longitudinal axis.

In one embodiment, upon application of a force to the lower member, the at least one angled protrusion passes over the ledge to permit the needle or blood bag tube to discharge liquid into the sampling container.

In one embodiment, the ledge is the upper edge of a resilient flange.

In one embodiment, the base member, the lower member and the upper member are made from a polymer.

Also disclosed herein is a sampling device adapted to connect in use a needle or blood bag tube with a sampling container; said device including; a base member having a bottom end, a side wall extending away from said bottom end to a rim to define a base chamber, an internal ledge on the internal perimeter of the base and an elongate boss located within said base chamber and extending away from said bottom end, said boss having an aperture defining a longitudinal axis; a spring adapted in use to be located at least partly within said chamber, about said boss and extending away from said bottom end; a lower member having a flange and an aperture at least one leg extending away from said flange opposite to said collar and an angled protrusion extending outwards from the longitudinal axis and wherein the angled projection sits above the internal ledge in a position wherein the needle or blood bag tube is not engaged with the sampling container; an upper member having a flange in use operatively associated with the flange of said lower member, said flange having an aperture corresponding with said aperture of said lower member, a boss extending about said aperture and away from said flange, a wall extending away from said flange to a rim to define a sampling chamber; at least one slot adapted in use to receive the at least one leg of said lower member in use, at least one further slot arranged to receive the angled protrusion in use; and a leg protrusion; whereby in use, said upper and lower members are adapted to move along said axis by the positioning of a sampling container in said sampling chamber and the application of a force on said lower member towards said base; upon application of force to the lower member, the angled protrusions pass over the internal projection, causing an end of said tube of said connector to extend beyond said boss of said upper member permitting a needle or blood bag tube located within said connector to discharge liquid into said sampling container.

In one embodiment, the internal ledge is part of a resilient member.

Also disclosed herein is a sampling device adapted to connect in use a needle or blood bag tube with a sampling container; said device including; a base member having a bottom end, a base rim, resilient extensions extending from the base rim, and an elongate boss located within said base chamber and extending away from said bottom end, said boss having an aperture defining a longitudinal axis; a spring adapted in use to be located at least partly within said chamber, about said boss and extending away from said bottom end; a lower member having a flange and an aperture at least one leg extending away from said flange opposite to said collar and an angled protrusion extending outwards from the longitudinal axis and wherein the angled projection sits above the resilient extensions in a position wherein the needle or blood bag tube is not engaged with the sampling container; an upper member having a flange in use operatively associated with the flange of said lower member, said flange having an aperture corresponding with said aperture of said lower member, a boss extending about said aperture and away from said flange, a wall extending away from said flange to an upper edge to define a sampling chamber; at least one slot adapted in use to receive the at least one leg of said lower member in use. whereby in use, said upper and lower members are adapted to move along said axis by the positioning of a sampling container in said sampling chamber and the application of a force on said lower member towards said base; upon application of force to the lower member, the angled protrusions deform the resilient extensions and pass over the resilient extensions, causing an end of said tube of said connector to extend beyond said boss of said upper member permitting a needle or blood bag tube located within said connector to discharge liquid into said sampling container.

### BRIEF DESCRIPTION OF DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 is a side view of a sampling device according to an embodiment of the present invention;

FIG. 2 is an exploded view of the sampling device of FIG. 1;

FIG. 3 is a partial cross sectional perspective view of an outer shell used in the sampling device of FIG. 1;

FIG. 4 is a perspective view of an outer shell used with the sampling device of FIG. 1;

FIG. 5 is a side view of the outer shell of FIG. 3;

FIG. 6 is a top down view of the outer shell of FIG. 3;

FIG. 7 is a partial cross sectional side view of the outer shell of FIG. 3;

FIG. 8 is a perspective view of an inner runner used in the sampling device of FIG. 1;

FIG. 9 is a perspective view of the inner runner of FIG. 8;

FIG. 10 is a side view of the inner runner of FIG. 8;

FIG. 11 is a cross sectional view of the inner runner of FIG. 8;

FIG. 12 is a top down view of the inner runner of FIG. 8;

FIG. 13 is a perspective view of a lower member used with the sampling device of FIG. 1;

FIG. 14 is a side view of the lower member of FIG. 13;

FIG. 15 is a side view of the lower member of FIG. 13;

FIG. 16 is a top down view of the lower member of FIG. 13;

FIG. 17 is a perspective view of a sampling receptacle for use with the sampling device of FIG. 1;

FIG. 18 is a top down view of the sampling receptacle of FIG. 19;

FIG. 19 is a partial cross sectional view of the sampling device of FIG. 1;

FIG. 20 is a partial perspective view of the sampling device of FIG. 1;

FIG. 21 is side view of a sampling device according to a second embodiment of the present invention;

FIG. 22 is a perspective view of the sampling device of FIG. 21;

FIG. 23 is a perspective view of a base of the sampling device of FIG. 21;

FIG. 24 is a perspective view of a base cover of the sampling device of FIG. 21;

FIG. 25 is a perspective view of the base cover of FIG. 24;

FIG. 26 is a perspective view of a lower member of the sampling device of FIG. 21;

FIG. 27 is a side view of the lower member of FIG. 26;

FIG. 28 is a perspective view of an upper member of the sampling device of FIG. 21;

FIG. 29 is a cross sectional perspective view of a receptacle engaged with the sampling device of FIG. 21;

FIG. 30 is cross sectional perspective view of a receptacle connected to, but in a disengaged position with the sampling device of FIG. 21;

FIG. 31 is a cross sectional perspective view of a receptacle engaged with a sampling device according to another embodiment of the present invention; and

FIG. 32 is a cross sectional perspective view of a receptacle connected in a disengaged position with the sampling of FIG. 31.

In the drawings like structures are referred to by like numerals throughout the several views. The drawings shown are not necessarily to scale, with emphasis instead generally being placed upon illustrating the principles of the present invention.

### DESCRIPTION OF EMBODIMENTS

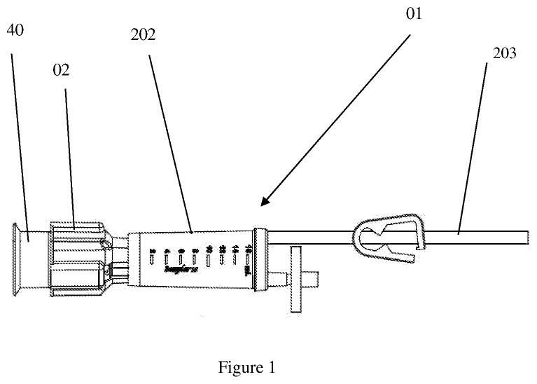

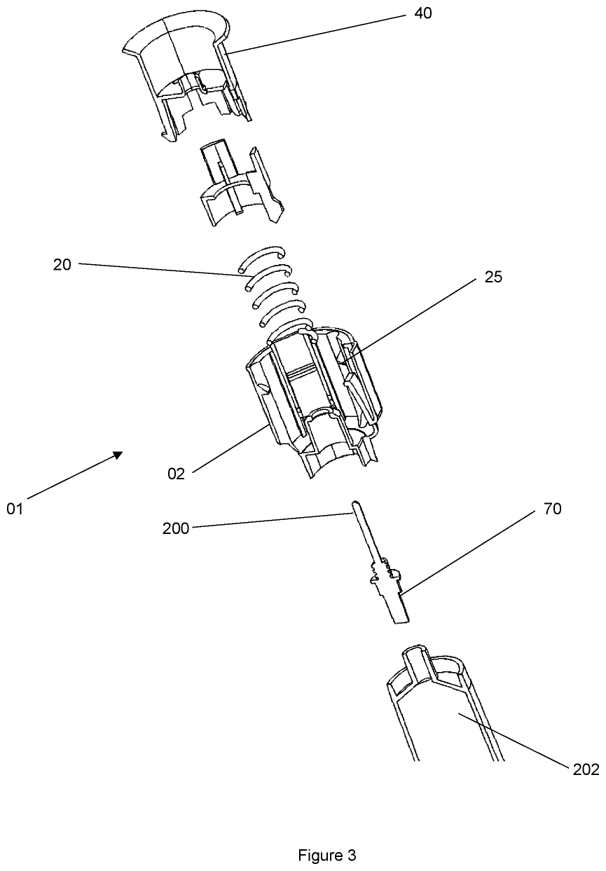

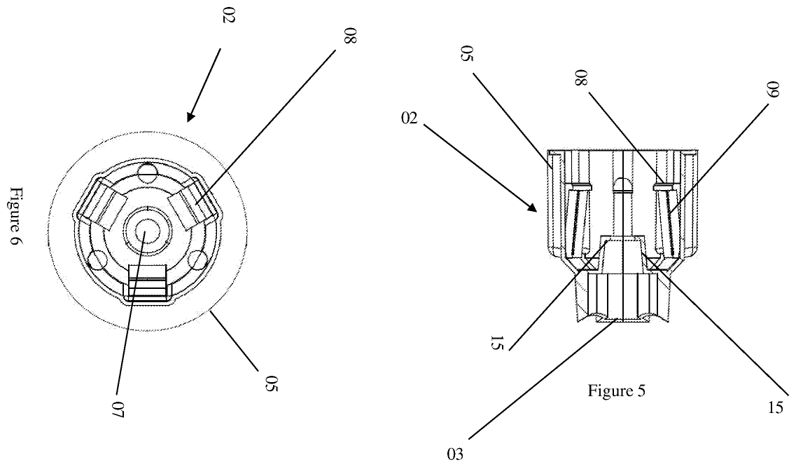

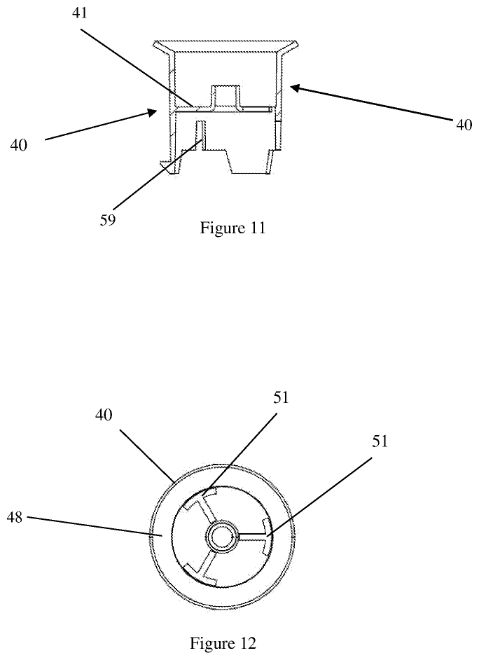

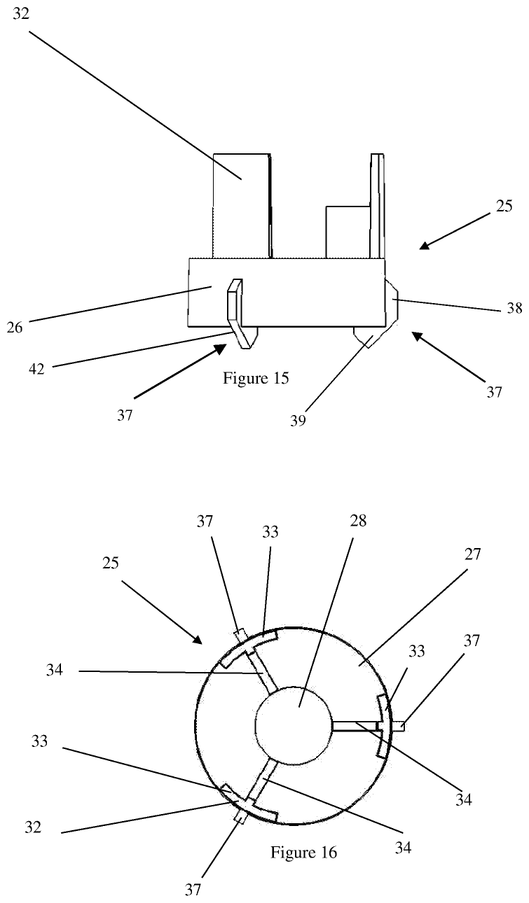

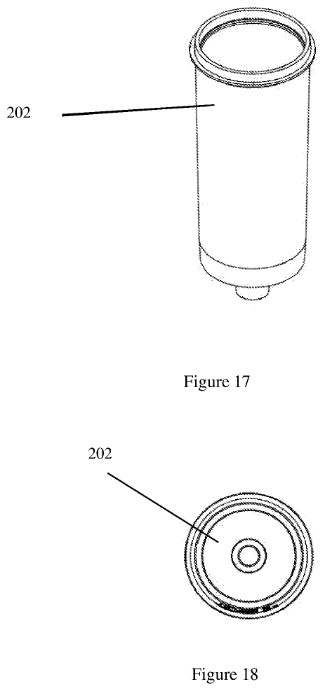

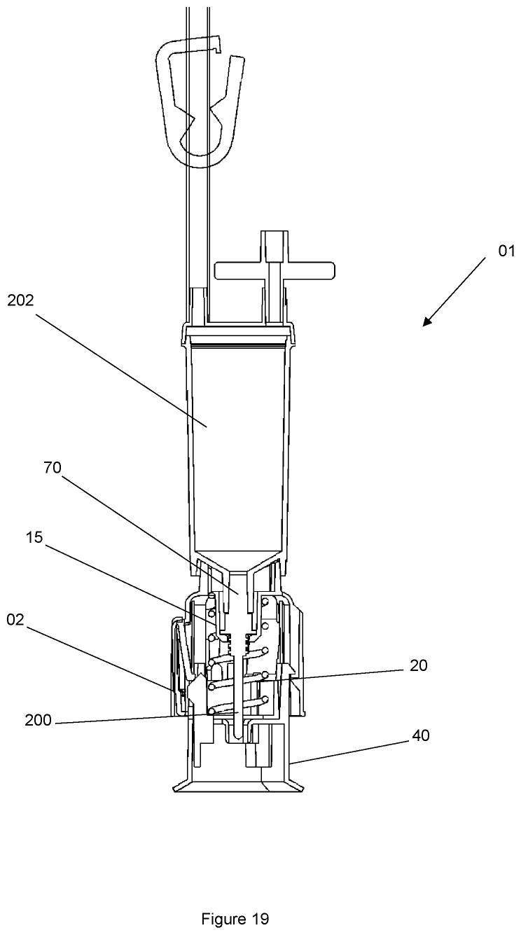

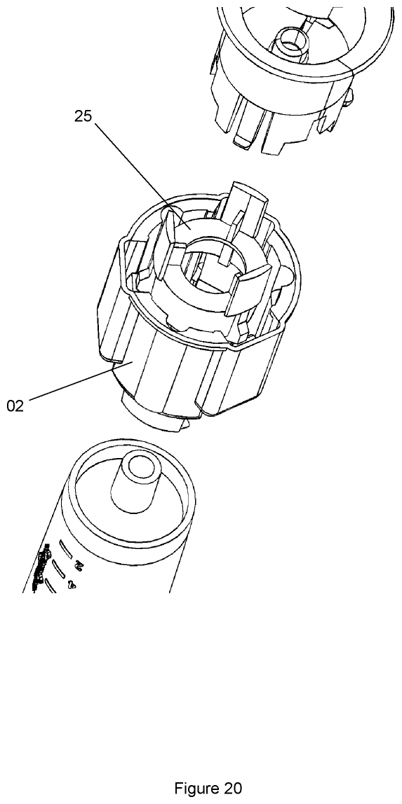

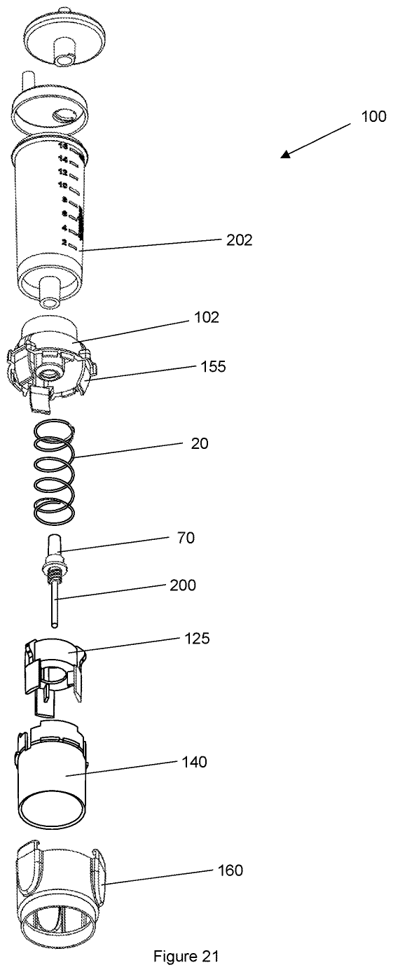

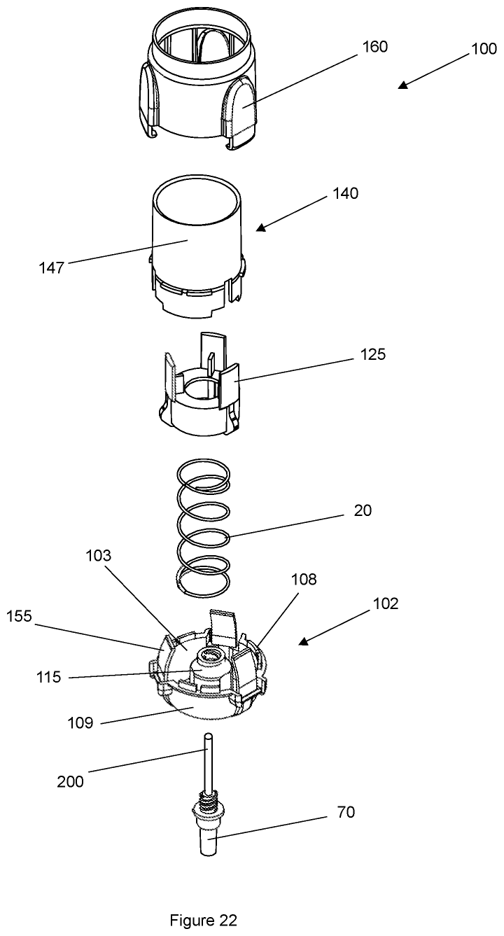

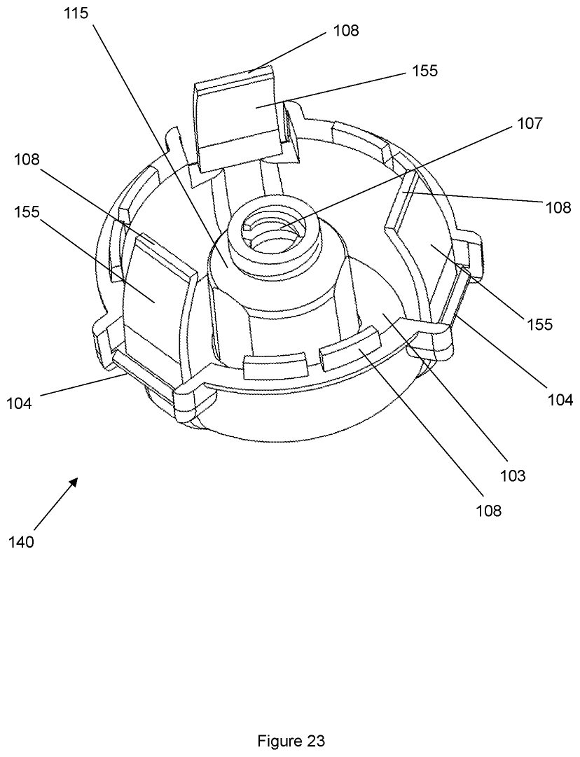

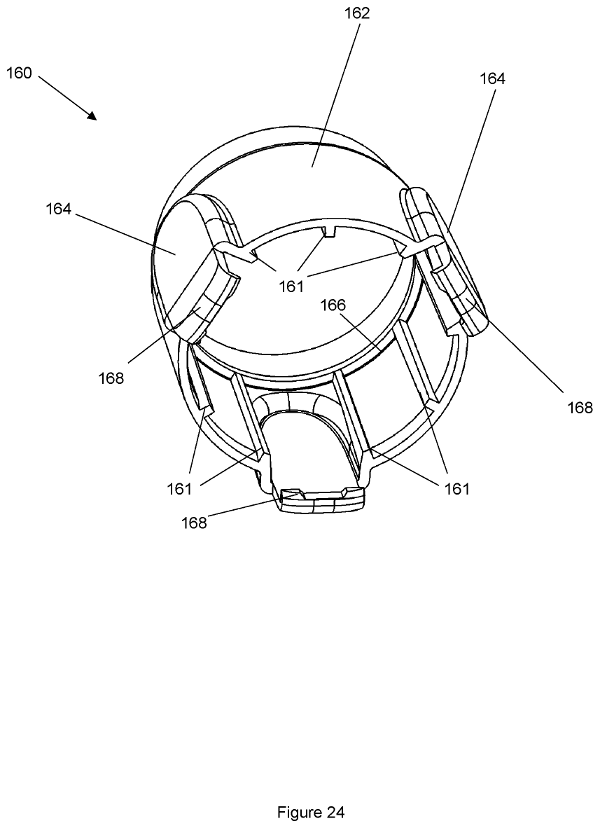

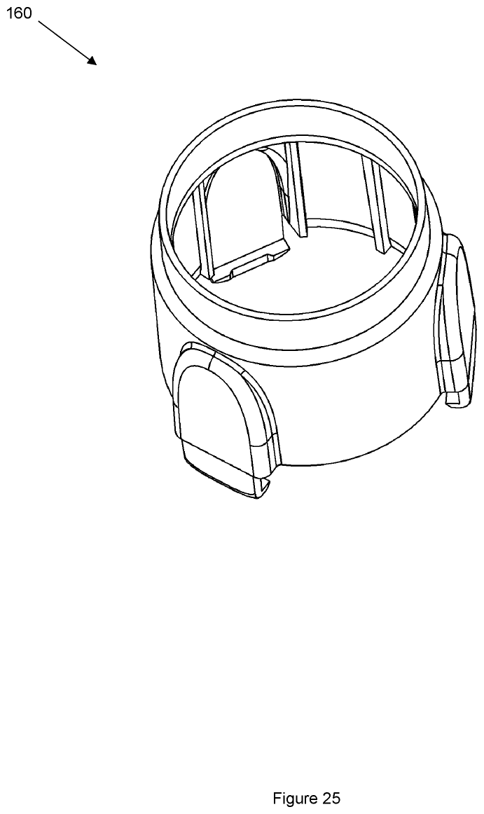

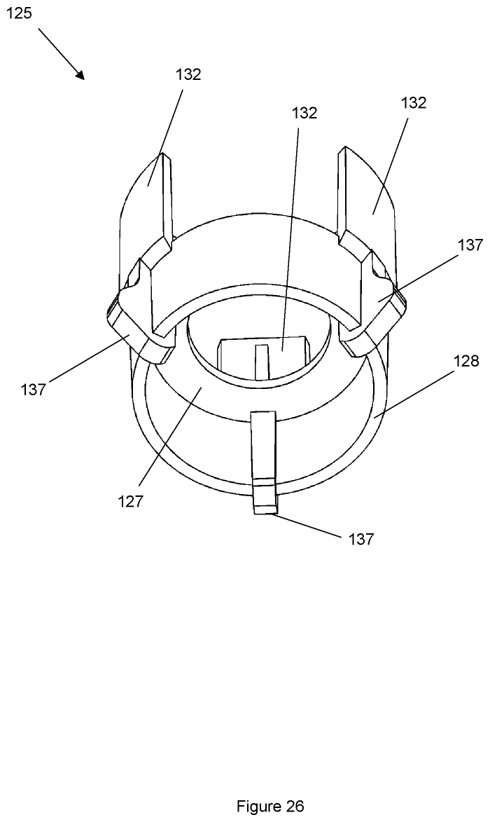

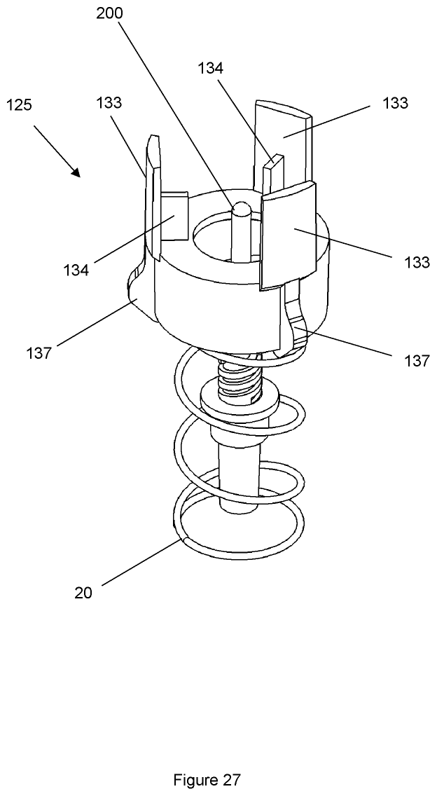

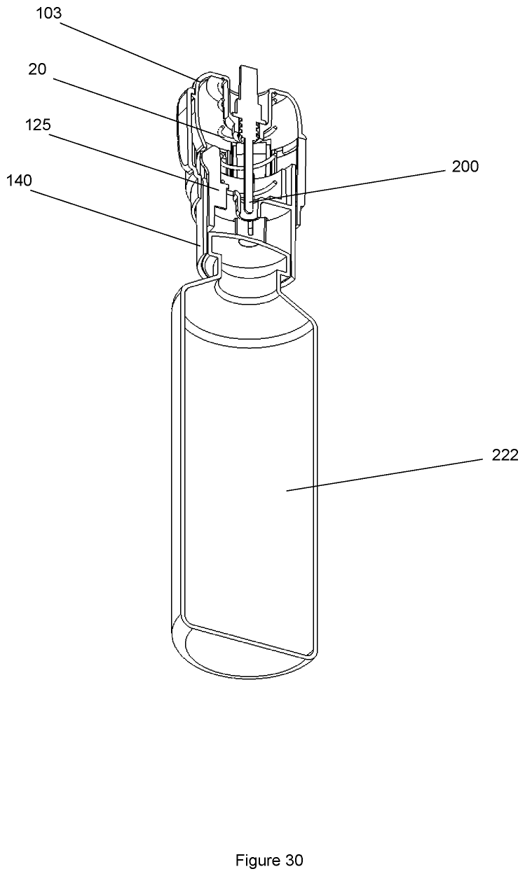

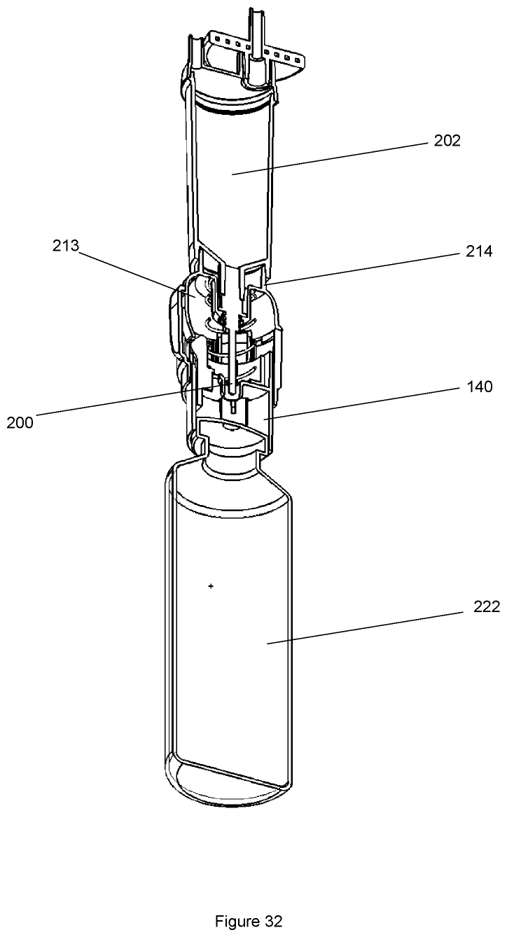

A first embodiment is shown in FIGS. 1 to 20 and includes a sampling device 01 adapted to connect a needle 200 with sampling containers 222 that have a variety of inlet/outlet profiles. The needle 200 and sampling containers 222 are shown in FIGS. 21 to 32.

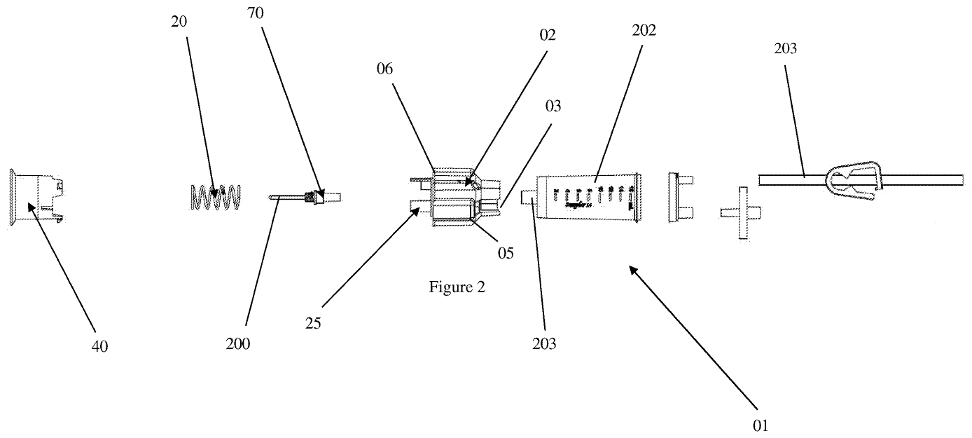

FIG. 2 shows the sampling device 01 in an exploded view. The sampling device 01 may be connected to a tube 203 from a blood bag or similar container without any needles involved. That is, the sampling device or adapter 01 is typically used by a doctor, researcher, healthcare worker, medical technician or the like to allow that person to collect samples of blood or body fluid either directly from a patient using venipuncture or directly from blood bags or similar containers. The needle 200 is often a fixed size of heavy gauge. The needle on the external end of the tube barrel holders may vary in gauze/size depending upon the patient.

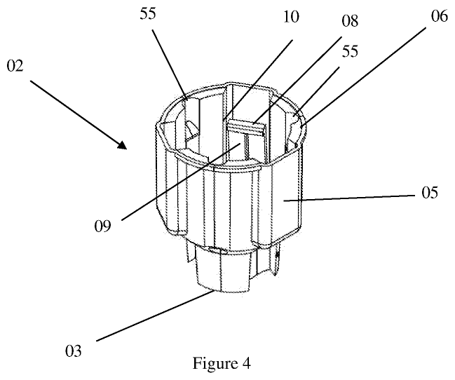

The sampling device 01 includes a base 02 more clearly shown in FIG. 4. The base 02 has a bottom end 03, a side wall 05 extending away from the bottom end 03 and towards a rim 06 to define a base chamber 10. In a preferred form, the base 02 is generally circular. However, it should be noted that there are other shapes such as squares, rectangles, or triangles that could also be utilised. The base 02 will be described in further detail below. An elongate boss 15 is located within the base chamber 10 and extends away from the bottom end 03.

A connector 70 is operatively associated with the boss 15 and the base 02 and has an elongated tube therethrough adapted to extend along a longitudinal axis of the sampling device 01 to receive the needle 200 and at the other end has suitable features to connect to a blood bag tubing, needles, luer needles or the like. Although commonly the connector 70 would take the form of a luer, it is also possible to have an assembly that does not include a luer taper but simply has a tube, connector or the like.

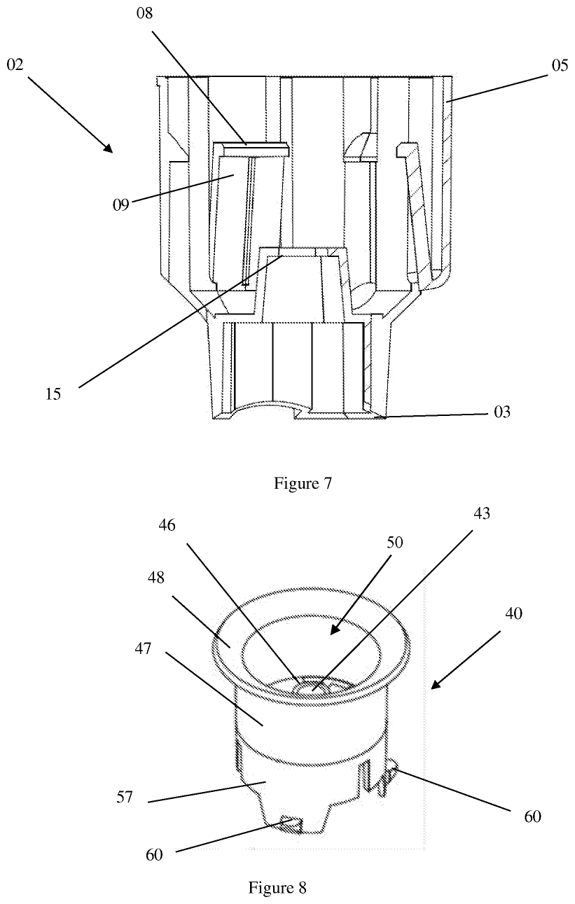

The boss 15 includes two steps in a preferred form and as best seen in FIG. 7. It could however, include many steps or only a single step. The boss 15 has an aperture 07 therethrough and as there are steps shown, the boss 15 has two different diameters. The aperture 07 could be the same diameter or different diameters within the boss 15. The longitudinal axis extends through aperture 07 along the longitudinal length of the sampling device 01. In an alternative embodiment (not shown), a portion or section of the aperture 07 in the boss 15 could be a connecting section and include a thread or the like to correspond with a connecting section on a connector inserted thereto. The connecting section though could include other fastening means such as bonding by an adhesive or the like. The sampling device 01 further includes a spring 20 adapted in use to be located at least partly within the base chamber 10 about the boss 15 and extending away from the bottom end 03. In an embodiment not shown in the drawings, a collar may extend from the bottom end 03 to retain or at least limit movement of the spring 20. The boss 15 is not to be read as being limited to having a circular profile.

In one embodiment, the upper region of the side wall 05, opening to the upper edge, includes channels 55. Channels 55 are arranged to receive protrusions 60 of lower member 25 described below.

The internal perimeter of the base 02 surrounding the base chamber 10 includes ledges 08 spaced apart from an upper edge of the base 02. Ledges 08 are the upper edge of a resilient member 09. Resilient member 09 is arranged to flex in the direction generally perpendicular to the extension of side wall 05.

In the figures, the outer surface 05 of the base 02 is illustrated as having a smaller diameter at the base 03 that flares out to a larger diameter in the upper region towards the rim 06. It is envisaged that the base may have a different external profile provided that the base chamber 10 receives the spring 20 and the lower member 25 in the manner as described throughout the specification.

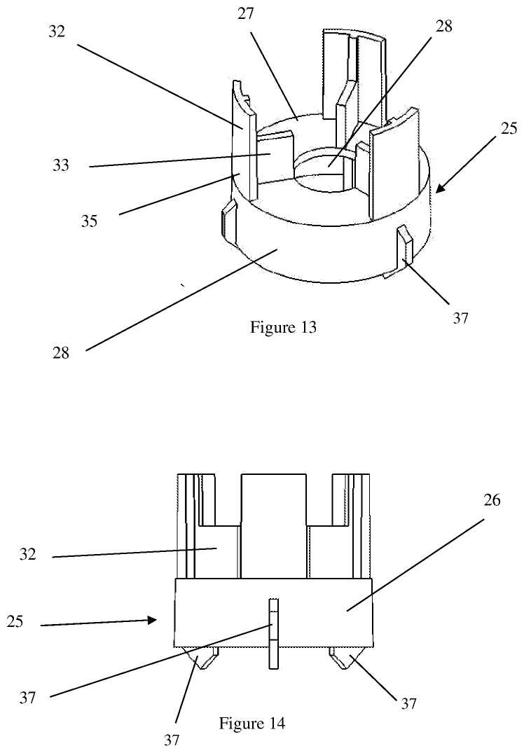

As best shown in FIGS. 13 to 16, lower member 25 is arranged to sit within the base chamber 10 in use, has a flange 27, an aperture 28, adapted in use to surround the longitudinal axis through aperture 17 along the longitudinal length of the sampling device 01, at least one leg 32 and angled protrusions 37 around the base perimeter 26 of the lower member 25. The at least one or more legs 32 extend away from the flange 27 opposite the angled protrusions 37. The legs comprise a first portion 33 that extends from the flange 27 around the perimeter of the flange 27 and second portion 34 that extends from the first portion 33 towards the centre of the flange 27. In the embodiments shown, there are four legs 32, however any number of legs 32 could be utilized.

It is envisaged that the legs 32 may be of a different arrangement on the flange 27 provided that their top down profile correspond with and aligns with slots 51 as described below.

In one embodiment not shown, the lower member includes a collar extending away from the flange 27 and adapted to retain or at least limit movement of an end of the spring 20.

The angled protrusion 37 extends outwards from the lower member bottom edge 28. The angled protrusion 37 includes a first portion 38 that extends outwards from the side of the lower member bottom edge 28 in a direction away from the centre of the flange 27. The angled protrusion 37 further includes a second portion 39 that extends outwards from lower member bottom edge 28 in a direction towards the base 02. An angled edge 42 extends between the first and second portions 38, 39.

In use, the angled protrusion 37 are above the ledges 08 in the base 03 to restrain movement of the lower member 25 towards the base 02. This restraint acts as a safety mechanism as the flange 27 of the lower member is restrained and will be described further below. It is envisaged that the angled protrusion takes an alternative profile that will work in the manner described for the present embodiment.

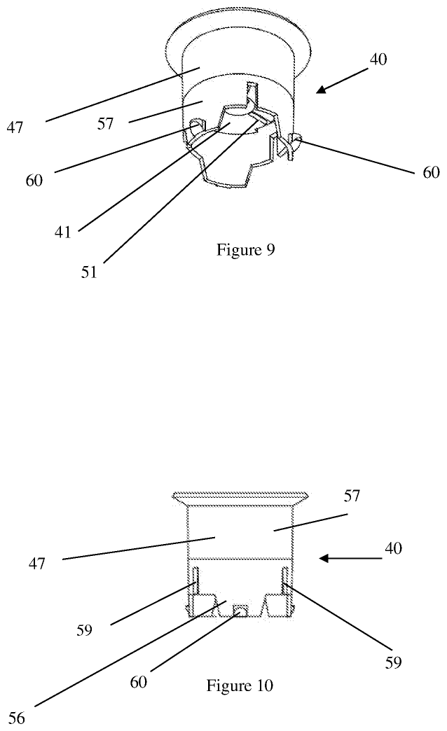

As best seen in FIGS. 8 to 12, there is shown an upper member 40 which has a separate flange 41 which in use is operatively associated with the flange 27 of the lower member 25. The upper member 40 is arranged to be received within the base chamber 10, being restrained by the base 02 in use. The upper member 40 includes lower wall 57 extending below the flange 41. Protrusions 60 project outwards from the base of legs 56 extending from the lower wall 57 and are arranged to engage corresponding inwards projections in channels 55 to restrain upwards movement of the upper member 40 when engaged to base 02. Use of alternative engagement means between the base 02 and the upper member 40 are within the scope of the present application. The upper member 40 acts as a safety shield to prevent accidental needle exposure. An aperture 43 is also located within the flange 41 which also corresponds to the aperture 28 of the lower member 25. A first projection 46 extends about that aperture 43 and away from the flange 41. An upper wall 47 extends away from the flange 41 to a rim 48 to define a sampling chamber 50. At least one slot 51 is provided in the flange 41 (as best seen in FIG. 9) and adapted in use to receive one or more legs 32 of the lower member 25 in use. In the preferred embodiment there are four legs 32 and four slots 51.

The spring 20 is designed to space the lower member 25 away from the base of the chamber 10, so that, in a neutral position, the needle 200 does not extend beyond the first projection 46 and in an active position, the space between the lower member 25 and the base of the chamber 10 is reduced to expose the needle 200 through aperture 43.

When assembled, the sampling device 01 includes the spring 20 in place between the bottom end 03 and the flange 27 of lower member 25. The protrusions 37 of lower member 25 sit above ledges 08 in base 02 and the projections 60 of upper member 40 pass through channels 55 and are restrained as described above.

Steps for engagement and disengagement of a sampling containers (generally a test tube or other similar container), typically include the sampling containers (for example a test tube) as it is cylindrical in shape and is sealed on one end and has an elastomeric bung on the other end. The sampling containers may be evacuated so as to have a lower pressure than atmospheric to allow the liquid to be “sucked” into the sampling containers. The elastomeric bung in the sampling containers is designed to be pierced by a needle 200 during transfer of liquid and automatically reseals when the needle 200 is retracted from the bung. In most cases, as the sheathed needle enters the bung, the sheath retracts and does not penetrate the bung, only the steel needle penetrates the bung. The connector 70 of the sampling device 01 is connected to a discharging container (for example a blood bag, arm of a patient or similar) via a suitable tubing or through a needle, cannula or other connectors like a blunt tip cannula. To transfer liquid from the discharging container. The sampling containers is inserted into the chamber 50 of the sampling device 01. In the first embodiment of the sampling device 01, the legs 32 of the lower member 25 protrude through slots 51 into chamber 50 and are pushed down when the sampling container is pushed towards the needle assembly (connector 70) as the rim of the sampling container engages the legs 32 applying force. The legs 32 by moving down cause the protrusion 37 to pass over the ledge 08 in a snap fit fashion, allowing the flange 41 of upper member 40 to move down over the sheathed needle and compressing the spring 20 in the process. The needle enters through the boss 15 in the lower member 25, through aperture 43 of first projection 46 and pierces a sheath and the elastomeric stopper of the sampling container and when fully penetrating the bung inside the sampling container allows the liquid to flow from the discharging container 202 into the sampling container via the connector 70 and the needle 200 inserted therein. When the required amount of liquid has been transferred into the receiving container, the sampling container is retracted out from the chamber 50. This causes the spring 20 to extend and push the lower and upper members 25, 40 back over the needle 200 with the protrusions 37 pushing out resilient members 09 to again be above the ledge 08. This process can be repeatable with the inclusion of an elastomeric sheath that retracts back over the needle 200 and seals the open end of the connector 70 thus stopping the flow of any further liquid until, if required, another sampling container is inserted into the sampling device 01 and the process repeated.

The projection 46 is not limited to being of a circular profile and can comprise a plurality of finger extensions around the aperture 43 or otherwise provided that the projection 46 fits within the aperture 43.

The sampling device 01 is designed to engage with vacuum tube caps that may have a variety of different cap designs for BD bottles or BACT/Alert bottle whilst preventing the ability of engagement with the needle 200 when a finger provides downward pressure. The ability of the upper member 40 to press towards the lower member 25 to come together requires that the projection 46 is free to pass through an aperture on the vacuum tube caps to be engaged with the sampling device 01 and that the vacuum tube engages and presses down the legs 32.

In accordance with a second embodiment, as shown in FIGS. 21 to 30 a sampling device 100 is illustrated. Features of the sampling device 100 that correspond to the features of the sampling device 01 of the first embodiment have been given the same numbering for illustrative purposes.

Broadly, the second embodiment of the present invention discloses an invention including a sampling device 100 adapted to connect a needle 200 with sampling containers 222 that have a variety of outlet profiles.

The second embodiment of the sampling device 100 of the present invention is described below.

The sampling device 100 includes a base 102 having a bottom end 103, and resilient extensions 155 extending away from the bottom end 103. The resilient extensions 155 are capable of flexing when force is applied to them in a direction along the longitudinal axis of the sampling device 100. In a preferred form, the base 102 is generally circular. However, it should be noted that there are other shapes such as squares, rectangles, or triangles that could also be utilised. The base 102 will be described in further detail below. An elongate boss 115 is located within the bottom end 103 inwards with respect to the sampling device 100 and extends away from the bottom end 103.

Connector 70 is operatively associated with the boss 115 and the base 102 and has an elongated tube therethrough adapted to extend along the longitudinal axis of the sampling device 100 receive needle 200 and at the other end has suitable features to connect to a blood bag tubing, needles, luer needles or the like. Although commonly the connector 70 would take the form of a luer, it is also possible to have an assembly that does not include a luer taper but simply has a tube, connector or the like.

The boss 115 includes two steps in a preferred form and as best seen in FIG. 24. It could however include many steps or only a single step. The boss 115 has an aperture 107 therethrough and as there are steps shown, the boss 115 has two different diameters. The aperture 107 could be the same diameter or different diameters within the boss 115. The aperture 107 defines a longitudinal axis through it along the longitudinal length of the sampling device 100. In an alternative embodiment (not shown), a portion or section of the aperture 107 in the boss 115 could be a connecting section and include a thread or the like to correspond with a connecting section on a connector inserted thereto. The connecting section though could include other fastening means such as bonding by an adhesive or the like. The sampling device 100 further includes a spring 20 adapted in use to be located at least partly against the bottom end 103 about the boss 115 and extending away from the bottom end 103. No limitations regarding a circular profile are to be read on to boss 115.

The resilient extensions 155 are curved towards the aperture 107.

In the figures, the base 102 is illustrated as having a generally circular profile. It is within the scope of the present invention for the base to have an alternative profile, such as rectangular, triangular or otherwise.

Sampling device 100 includes a lower member 125. For sampling device 100 lower member 125 is arranged to sit against the bottom end 103 of the base 102 around the boss 115. Lower member 125 has a flange 127, an aperture 128, adapted in use to surround the longitudinal axis through aperture 117 along the longitudinal length of the sampling device 100, at least one leg 132 and angled protrusions 137 around the base perimeter 126 of the lower member 125. The at least one or more legs 132 extend away from the flange 127 opposite the angled protrusions 137. The legs comprise a first portion 133 that extends from the flange 127 around the perimeter of the flange 127 and second portion 134 that extends from the first portion 133 towards the centre of the flange 127. In the embodiments shown, there are three legs 132, however alternative number of legs 132 could be utilized.

In one embodiment, the edge of the flange 127 surrounding the aperture 128 and intersects with the second portion 134 includes a recess 140 arranged to receive the diameter of the spring 20 wire.

The angled protrusion 137 extends outwards from the lower member bottom edge 128. The angled protrusion 137 includes a first portion 138 that extends outwards from the side of the lower member bottom edge 128 in a direction away from the centre of the flange 127. The angled protrusion 137 further includes a second portion 139 that extends outwards from lower member bottom edge 128 in a direction towards the base 102. An angled edge 142 extends between the first and second portions 138, 139.

In use, the angled protrusion 137 are situated above the top edges 108 of the resilient extensions 155. In sitting above the top edge 108, in a neutral situation, the lower member 125 is restrained from movement towards the bottom end 103 by the resilient extensions 155. This restraint acts as a safety mechanism as the flange lower member is restrained and will be described further below. It is within the scope of the present invention that the angled protrusion 137 takes an alternative profile that will work in the manner described for the present embodiment.

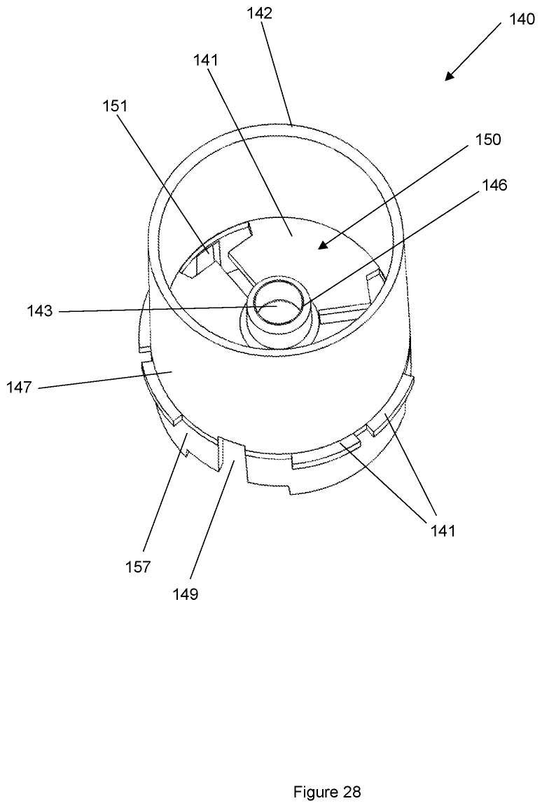

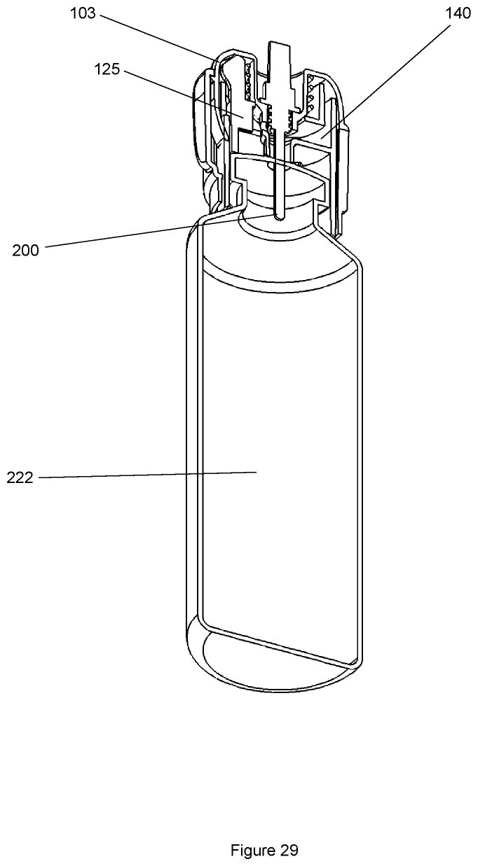

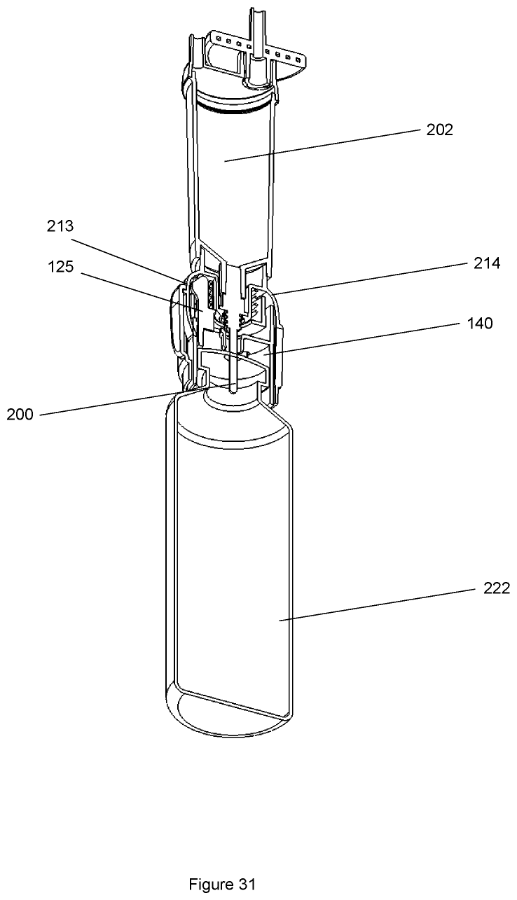

As best seen in FIG. 28, there is shown an upper member 140 which has a wall 142 and a separate flange 141 which in use is operatively associated with the flange 127 of the lower member 125. The upper member 140 is arranged to be received within a base cover 160 that is described further below. The upper member 140 includes lower wall 157 extending below the flange 141. Protrusions 161 project outwards from an upper region of the lower wall 157 and engage with base cover 160 as set out below. The upper member 140 acts as a safety shield to prevent accidental needle exposure. An aperture 143 is also located within the flange 141 which also corresponds to the aperture 128 of the lower member 125. A protrusion 146 extends about that aperture 143 and away from the flange 141. An upper wall 147 extends away from the flange 41 to an upper edge to define a sampling chamber 150. At least one slot 151 is provided in the flange 141 (as best seen in FIG. 31) and adapted in use to receive one or mor legs 132 of the lower member 125 in use. In the preferred embodiment there are four legs 132 and four slots 151.

The lower wall 157 includes a slot arranged for the protrusion 137 to rest in use.

As best shown in FIGS. 24 and 25, the sampling device 100 includes a base cover 160 arranged to connect to the base 102 and the upper member 140 described below. The base cover 160 includes a side wall 162 with a rim 166 at its upper edge. The side walls include recessed portions 164 that protrude from the perimeter of the base cover 160. The recess portions 164 include a clip extension 168 extending from the base of the base cover 160. The clip extension 168 extends to engage with base 102 and restrain the upper member 140, lower member 125, spring 20 and base 102 together. The clip 168 is flexible and is arranged to pass over the resilient extensions 155 and over the base edge 104 of the resilient extensions 155 to clip the base cover 160 and the base 102 together. In one embodiment, the clip extension 168 locks to the base edge 104 through snap lock means.

It is withing the scope of the present invention that the clip extension 168 locks to the base edge 104 through alternative means, such as screw fit, bolting or otherwise as understood by the skilled addressee.

The base cover 160 is arranged to so that the protrusions 161 of the upper member 140 sit underneath, and are restrained by the upper rim 166 of the base cover 160. The rim 166 allows movement of the upper member 140 away from the rim 160 towards the clip extensions 168, but restrains movement of the protrusions 161 past the rim 166 away from the clip extensions 168.

The spring 20 is designed to space the lower member 125 away from the base of the chamber 10, so that, in a neutral position, the needle 200 does not extend beyond the second projection 46 and in an active position, the space between the lower member 125 and the base of the chamber 110 is reduced to expose the needle 200.

When assembled, the sampling device 100 includes the spring 20 in place between the bottom end 103 and the flange 127 of lower member 125. The protrusions 137 sit above upper edges 108 of resilient extensions 155 base cover 160 engages the upper member 140 and lower member as described above.

Steps for engagement and disengagement of a sampling containers (generally a test tube or other similar container), typically include the sampling containers (for example a test tube) as it is cylindrical in shape and is sealed on one end and has an elastomeric bung or on the other end. The sampling containers may be evacuated so as to have a lower pressure than atmospheric to allow the liquid to be “sucked” into the sampling containers. The elastomeric bung in the sampling containers is designed to be pierced by a needle 200 during transfer of liquid and automatically reseals when the needle 200 is retracted from the bung. In most cases, as the sheathed needle enters the bung, the sheath retracts and does not penetrate the bung, only the steel needle penetrates the bung. The connector 70 of the sampling device 100 is connected to a discharging container (for example a blood bag, arm of a patient or similar) via a suitable tubing or through a needle, cannula or other connectors like a blunt tip cannula. To transfer liquid from the discharging container. The sampling containers is inserted into the chamber 150 of the sampling device 100. In the first embodiment of the sampling device 100, the legs 132 of the lower member 125 protrude through slots 151 into chamber 150 and are pushed down when the sampling container is pushed towards the needle assembly (connector 70) as the rim of the sampling container engages the legs 32 applying force. The first and second portions 133 and 134 ensure that different sized sampling containers engage the legs 32. The legs 32, by moving down on the application of force cause the protrusion 37 to push the curved resilient extensions towards the aperture 107 allowing the protrusions 137 and the lower member 125 to pass beyond the upper edge 108 of the resilient member 155 to the bottom end 103, causing the space between the bottom end 103 and flanges 141 to decrease. The needle enters through the boss 115 in the lower member 125 and pierces a sheath and the elastomeric stopper of the sampling container and when fully penetrating the bung inside the sampling container allows the liquid to flow from the discharging container 202 into the sampling container via the connector 70 and the needle 200 inserted therein. When the required amount of liquid has been transferred into the receiving container, the sampling container is retracted out from the chamber 150. This causes the spring 20 to extend and push the lower and upper members 125, 140 back over the needle 200 with the protrusions 137 pushing out resilient extensions 155, away from aperture 143 to again be above the upper edge 08. This process can be repeatable with the inclusion of an elastomeric sheath that retracts back over the needle 200 and seals the open end of the connector 70 thus stopping the flow of any further liquid until, if required, another sampling container is inserted into the sampling device 100 and the process repeated.

The protrusion 146 is not limited to being of a circular profile and can comprise a plurality of finger extensions around the aperture 143 or otherwise provided that the protrusion 146 fits within the aperture 143.

The sampling device 100 is designed to engage with the vacuum tubes caps having a variety of different cap designs for BD bottles or BACT/Alert bottle whilst preventing the ability of engagement with the needle 200 when a finger provides downward pressure. The ability of the upper member 140 to press towards the lower member 125 to come together requires that the projection 146 is free to pass through an aperture on the vacuum tube caps to be engaged with the sampling device 100 and that the vacuum tube engages and presses down the legs 32.

Although the invention has been described with reference to specific examples, it will be appreciated by those skilled in the art that the invention may be embodied in many other forms.

1. A sampling device adapted to connect in use a needle or blood bag tube with a sampling container, the sampling device configured for movement between a neutral position and an active position, the sampling device including:
a base member including:
at least one base member sidewall to define a base chamber; and
a boss located within the chamber, the boss including a base member aperture configured for holding the needle in use;
a lower member configured for nesting at least partially within the base chamber, the lower member including:
a lower member aperture axially aligned with the base member aperture to receive the needle therethrough;
at least one lower member leg that extend from around the lower member aperture to engage the sampling container in use;
an upper member configured for axial movement toward and away from the base member, the upper member including:
at least one upper member sidewall to define an upper member chamber;
an upper member protrusion located within the upper member chamber, the upper member protrusion including an upper member aperture axially aligned with the base member aperture for the needle to pass therethrough; and
a spring seated on the boss, the spring configured for biasing the lower member away from the base member toward the neutral position,
wherein in the neutral position, the upper member protrusion is configured to shield the needle, and in the active position the needle is exposed from the upper member protrusion and shielded by the upper member chamber.

2. The sampling device of claim 1, wherein the at least one lower member leg is configured to engage the sampling container when the sampling container is inserted into the upper member chamber.

3. The sampling device of claim 2, wherein when the at least one lower member leg is seated on the sampling container, and the sampling device is moved toward the sampling container such that the sampling device moves from neutral position to the active position, the needle protrudes from the upper member protrusion.

4. The sampling device of claim 3, wherein the sampling device is movable from the neutral position to the active position when the at least one lower member leg is seated on a shoulder of the sampling container.

5. The sampling device of claim 4, wherein the lower member includes a flange, the at least one lower member leg extends from the flange.

6. The sampling device of claim 5, wherein a longitudinal axis extends through the base member aperture, the lower member aperture and the upper member aperture.

7. The sampling device of claim 6, wherein the upper member includes at least one slot adapted in use to receive the at least one lower member leg.

8. The sampling device of claim 7, wherein at least one slot is transverse to the longitudinal axis to allow the at least one lower member leg to pass therethrough.

9. The sampling device of claim 8, wherein the lower member includes at least one angled protrusion extending outwards from the longitudinal axis.

10. The sampling device of claim 9, wherein the upper member includes at least one further slot arranged to receive the angled protrusion in use.

11. The sampling device of claim 10, wherein the base member sidewall includes a ledge extending toward the longitudinal axis.

12. The sampling device of claim 11, wherein upon application of a force to the lower member, the at least one angled protrusion passes over the ledge to permit the needle or blood bag tube to discharge liquid into the sampling container.

13. The sampling device of claim 12, wherein the ledge is the upper edge of a resilient flange.

14. The sampling device of claim 13, wherein the base member, the lower member and the upper member are made from a polymer.

15. A sampling device adapted to connect in use a needle or blood bag tube with a sampling container; said device including:
a base member having a bottom end, a side wall extending away from said bottom end to a rim to define a base chamber, an internal ledge around the internal perimeter of the base and an elongate boss located within said base chamber and extending away from said bottom end, said boss having an aperture through which a longitudinal axis extends;
a spring adapted in use to be located at least partly within said chamber, about said boss and extending away from said bottom end;
a lower member having a flange, an aperture and at least one leg extending away from said flange opposite and an angled protrusion extending outwards from the longitudinal axis and wherein the angled projection sits above the internal ledge in a position wherein the needle or blood bag tube is not engaged with the sampling container;
an upper member having a flange in use operatively associated with the flange of said lower member, said flange having an aperture corresponding with said aperture of said lower member, a first projection extending about said aperture and away from said flange, a wall extending away from said flange to a rim to define a sampling chamber; at least one slot adapted in use to receive the at least one leg of said lower member in use, at least one further slot arranged to receive the angled protrusion in use; and a leg protrusion;
at least one further slot arranged to receive the angled protrusion in use; and a leg protrusion;
whereby in use, said upper and lower members are adapted to move along said axis by the positioning of a sampling container in said sampling chamber and the application of a force on said lower member towards said base; upon application of force to the lower member, the angled protrusions pass over the internal projection, causing an end of said tube of said connector to extend beyond said first projection of said upper member permitting a needle or blood bag tube located within said connector to discharge liquid into said sampling container.

16. The sampling device of claim 1, wherein the internal ledge is the upper edge of a resilient flange.

17. A sampling device adapted to connect in use a needle or blood bag tube with a sampling container; said device including;
a base member having a bottom end, a base rim, resilient extensions extending from the base rim, and an elongate boss located within said base chamber and extending away from said bottom end, said boss having an aperture through which a longitudinal axis extends;
a spring adapted in use to be located at least partly within said chamber, about said boss and extending away from said bottom end;
a lower member having a flange and an aperture at least one leg extending away from said flange opposite to said collar and an angled protrusion extending outwards from the longitudinal axis and wherein the angled projection sits above the resilient extensions in a position wherein the needle or blood bag tube is not engaged with the sampling container;
an upper member having a flange in use operatively associated with the flange of said lower member, said flange having an aperture corresponding with said aperture of said lower member, a second projection extending about said aperture and away from said flange, a wall extending away from said flange to an upper edge to define a sampling chamber;
at least one slot adapted in use to receive the at least one leg of said lower member in use whereby, said upper and lower members are adapted to move along said axis by the positioning of a sampling container in said sampling chamber and the application of a force on said lower member towards said base; upon application of force to the lower member, the angled protrusions deform the resilient extensions and pass over the resilient extensions, causing an end of said tube of said connector to extend beyond said second projection of said upper member permitting a needle or blood bag tube located within said connector to discharge liquid into said sampling container.
